(19) United States
(12) Patent Application Publication
(10) Pub. No.: US 2014/0249405 A1
(43) Pub. Date: Sep. 4, 2014

(54) IMAGE SYSTEM FOR PERCUTANEOUS INSTRUMENT GUIDENCE

(71) Applicant: IGIS INC., Half Moon Bay, CA (US)

(72) Inventor: John Savage WIMER, Half Moon Bay, CA (US)

(73) Assignee: IGIS INC., Half Moon Bay, CA (US)

(21) Appl. No.: 13/956,700

(22) Filed: Aug. 1, 2013

Related U.S. Application Data
(60) Provisional application No. 61/771,755, filed on Mar. 1, 2013.

(51) Int. Cl. A61B 5/06; A61B 8/08; G01R 33/28; A61B 6/12

(52) U.S. Cl. CPC A61B 5/066; A61B 6/12; A61B 8/0841; G01R 33/285
USPC 600/424

(57) ABSTRACT

Hardware and software methodology are described including a synthesized User Interface (UI) to provide guidance for injection and other procedures under medical imaging. The UI provides a practitioner the option of viewing multiple images concurrent with a live image. A guidance image corresponds to correct probe placement for a selected procedure, a reference image corresponds to an expected view with such probe placement in the live image. The reference image may be variously labeled. Another option involves probe tracking to update the guidance and/or reference image view(s).

### CROSS REFERENCE WITH RELATED APPLICATION

This application claims the benefit of U.S. Provisional Application No. 61/771,755, filed on Mar. 1, 2013. The above-referenced application is hereby incorporated by reference in its entirety for all purposes.

### TECHNICAL FIELD

This filing relates to image systems for aiding instrument/instrumentation insertion in desired anatomical locations. More specifically, it relates to improved instructional tools for practitioners as well as improved confirmation of instrument location and placement related to anatomical targeting.

### BACKGROUND OF THE INVENTION

With increased pressure on medical practitioners to generate revenue, document therapy/treatment and improve patient outcome, there is a need for imaging tools to facilitate the same.

By way of example, it has been noted by several expert orthopedists that only about 70% of therapeutic injections are effective. One such treatment is steroid injection for tennis elbow. When a first injection is unsuccessful at remedying the pain, subsequent injections are often employed to reduce or eliminate the pain. When visualization is utilized for needle placement, the practitioner can be sure of the therapeutic application.

A majority of general practitioners and orthopedists do not have “handy” imaging systems available for performing such procedures. They sometimes also lack the experience to readily recall optimal means of utilizing the tools that are available to them or the specifics of anatomical landmarks in the images generated. Rather, many physicians rely on memory of text books for what to look for on images generated by x-ray, ultrasound, MRI, etc. In addition, physicians generally have very little to assist in placement of the patient or of the imaging device itself to generate a useful image.

Accordingly, a need exists for improved instrument location and anatomical targeting/confirmation tools for physicians. Aspects of the present invention meet these needs and others as will be apparent to those with skill in the art in review of the subject disclosure.

### SUMMARY

The inventive embodiments include devices and systems (e.g., including the sensor and display hardware referenced herein, the addition of a computer processor and other ancillary/support electronics and various housing elements) and methods (including the hardware and software for carrying out the same), addressing the features described herein. Such methods and devices are adapted for percutaneous instrument guidance.

While there are many tools available for basic imaging, a synthesized User Interface (UI) is provided for enabling improved outcomes. The guidance provided thereby may be especially useful for a non-expert practitioner. However, the systems' utility is not so-limited. The subject UI provides any practitioner the option of viewing multiple images concurrent with a live image.

At minimum, three UI images may be provided. A guidance image corresponds to correct probe placement for a selected procedure, a reference image corresponds to an expected view with such probe placement, and the third image is the live image which should bear strong resemblance to the expected view while undertaking the medical procedure. The guidance and/or reference image may be variously labeled. Another option involves probe tracking to update the guidance and/or reference image view(s).

In a dental application, a technician would select the desired shots and an image of x-ray arm placement would be displayed showing how to place the arm against the patient's mandible or maxilla. In addition, a standard x-ray image could also be displayed.

In a musculoskeletal application in which ultrasound imaging is to be utilized, a menu selection for the desired anatomical location is made. An image on a display screen shows proper placement of the transducer and what an ultrasound image should look like. Landmarks inside of the image may also be labeled and placement of an instrument could be demonstrated. These all assist the practitioner with effecting an optimal treatment for the subject/patient.

The subject imaging systems, a compilation (or table) of images is provided matching the device being utilized. For instance, a system made for an MRI manufactured by Siemens, Inc. would have images generated by Siemens equipment. For a system including an Interson, Inc. transducer, the images provided and generated correspond to the Interson transducer.

In one embodiment, an ultrasound transducer/probe is attached to a computer. When the processing software application is initiated, it senses the probe and may display comparative image(s) matching the probe. The user then typically makes a selection for the anatomy of interest. The system displays an example of the correct image to be achieve for treating the selected anatomy (i.e., reference image), along with an image of correct transducer placement (i.e., guidance image) for achieving such an image. Such images may be still/singular images. Or they may be animated clips or movies or medical imaging “cines” as the case may be. Stated otherwise, the examples of correct image acquisition device orientation/placement and samples of anatomical images can be proved in sequence or simultaneously. Generally, the pictures/illustrations/images may be in 2D, 3D, or 4D-moving or still.

Likewise, an optional aspect is for the image acquisition device image (e.g., ultrasound transducer) to display a 2 dimensional image that can be rotated on screen so as to appear 3 dimensional, thereby showing from a variety of points of perspective the transducer placement. Additionally, once the orientation of the subject (subject/patient) is locked in place in the guidance image, the transducer itself can be manipulated. This in turn can be synchronized with the guidance and/or reference image for tracking orientation in unison. If the entire guidance image is locked, then the reference image can be run as a video showing instrument (i.e., needle placement and injection) deployment. Likewise, software monitoring of motion sensors in the transducer can be utilized to manipulate the reference image(s). For instance, if one were viewing longitudinally, the transducer could be rotated approximately 90 degrees to a transverse view and that motion would trigger the reference image view to shift accordingly.

In addition to manual adjustments made to the computer, instruction to the control software can be given verbally and translated by voice recognition incorporated into the software for control purposes. Indeed, every aspect of the software may be controlled by voice commands. Otherwise, a touch-screen tablet interface may be convenient.

Users of the subject systems may include any of spine and orthopedic surgeons and specialists, general practitioners, radiologists and interventional neuroradiologists, neurosurgeons, sonographers, physiatrists, pain management specialists and/or rheumatologists. Target locations and treatments may include any of the synovium (by injections or aspirations for synovitis), bursae (by injections or aspirations for superficial and deep bursitis), tendons/ligaments (by injections for tendonitis or tenosynovitis), cartilage (by injections for palliative treatment of cartilage defects and calcification), muscle (by injections for muscle trauma), spine (for epidural injection) and joints (by injections for palliative treatment of joint erosion). Joints to be targeted may include any of the shoulder, elbow, wrist/hand, hip, knee and/or ankle/foot.

In use, the subject systems offer the potential for improved musculoskeletal injection and other percutaneous procedure accuracy. Given its advantages, the systems may support a trend in procedure conversion from specialists to generalists such that general practitioner and orthopedic physicians can expertly perform injections instead of referring-out the work (since the sonographer/radiologist typically required for imaging with existing hardware solutions is not needed). Such advantages are optionally realized with low cost, yet high performance systems as further described herein. In other words, the subject systems can be economically produced and made available for less cost than known imaging systems that are generally regarded as not user-friendly.

### BRIEF DESCRIPTION OF THE DRAWINGS

The figures provided herein may be diagrammatic and not necessarily drawn to scale, with some components and features exaggerated and/or abstracted for clarity. Variations from the embodiments pictured are contemplated. Accordingly, depiction of aspects and elements in the figures are not intended to limit the scope of the claims, except when such intent is explicitly stated.

FIG. 1 is an overview of the subject system.

FIG. 2 represents a basic UI example.

FIG. 3 represents a more refined UI example.

FIG. 4 is a software process flowchart.

FIGS. 5A and 5B depict further UI options.

### DETAILED DESCRIPTION

Various example embodiments are described below. Reference is made to these examples in a non-limiting sense. They are provided to illustrate more broadly applicable aspects of inventive aspects. Various changes may be made to the embodiments described and equivalents may be substituted without departing from their true spirit and scope. In addition, many modifications may be made to adapt a particular situation, material, composition of matter, process, process act(s) or step(s) to the objective(s), spirit or scope of the claims made herein.

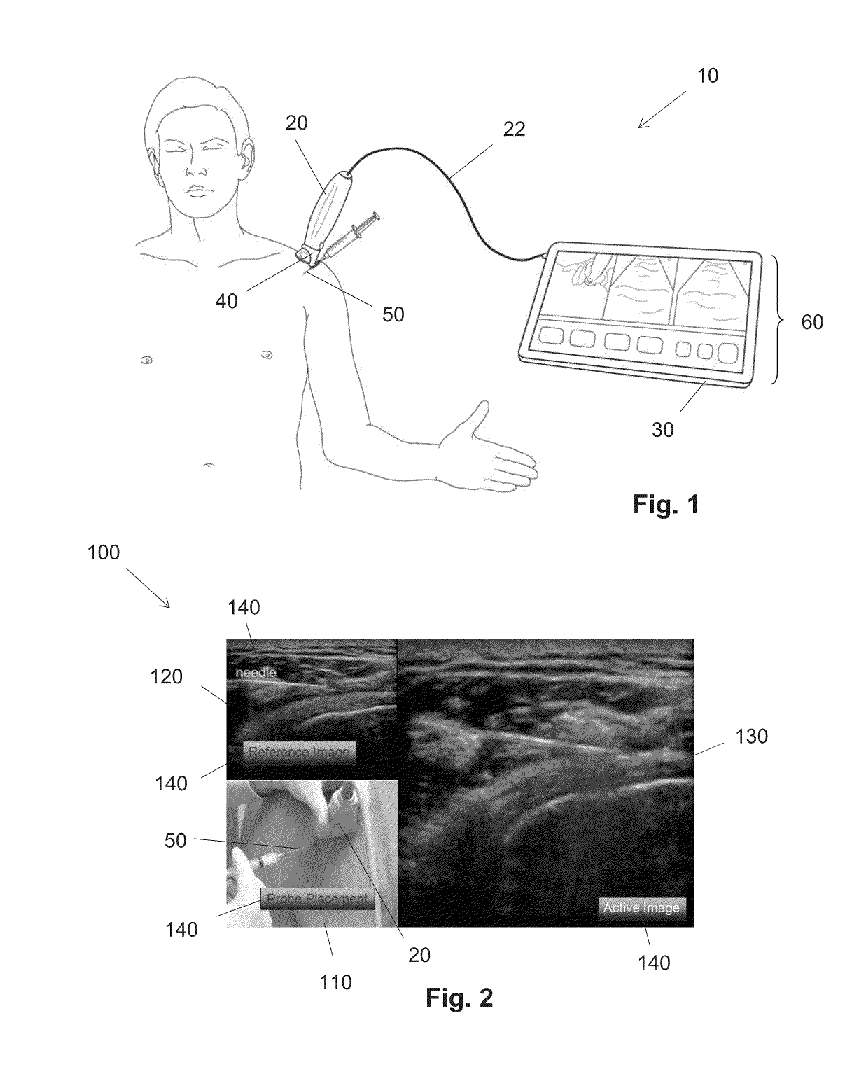

That said, an exemplary system 10 is shown in FIG. 1. For treating a patient, system 10 may include a mid-range ultrasound depth probe 20 (for 0.5-6.0 em imaging) and a tablet computer 30 (e.g., an IPAD) running the subject software. A suitable probe is produced by Interson, Inc., under one or more of U.S. Pat. Nos. 6,099,474 or 8,114,024 or US Patent Publication No. 2007/0239019. A USB type cable 22 interface may connect the probe to the computer. Further, an assortment of sizes of needle guides (single guide 40 shown) may be provided along with matching assortment of echogenic needles (single needle 50 shown). A practice phantom (not shown) may be packaged in a kit with several of the noted components. Likewise, additional equipment and supplies such as a shallow depth probe (0.0-2.0 em) a deep probe (3.0-15.0 em) and/or a probe standoff(not shown) may be provided. Software running on the computer device provides a User Interface (UI) 60 embodying various optional inventive features as detailed below.

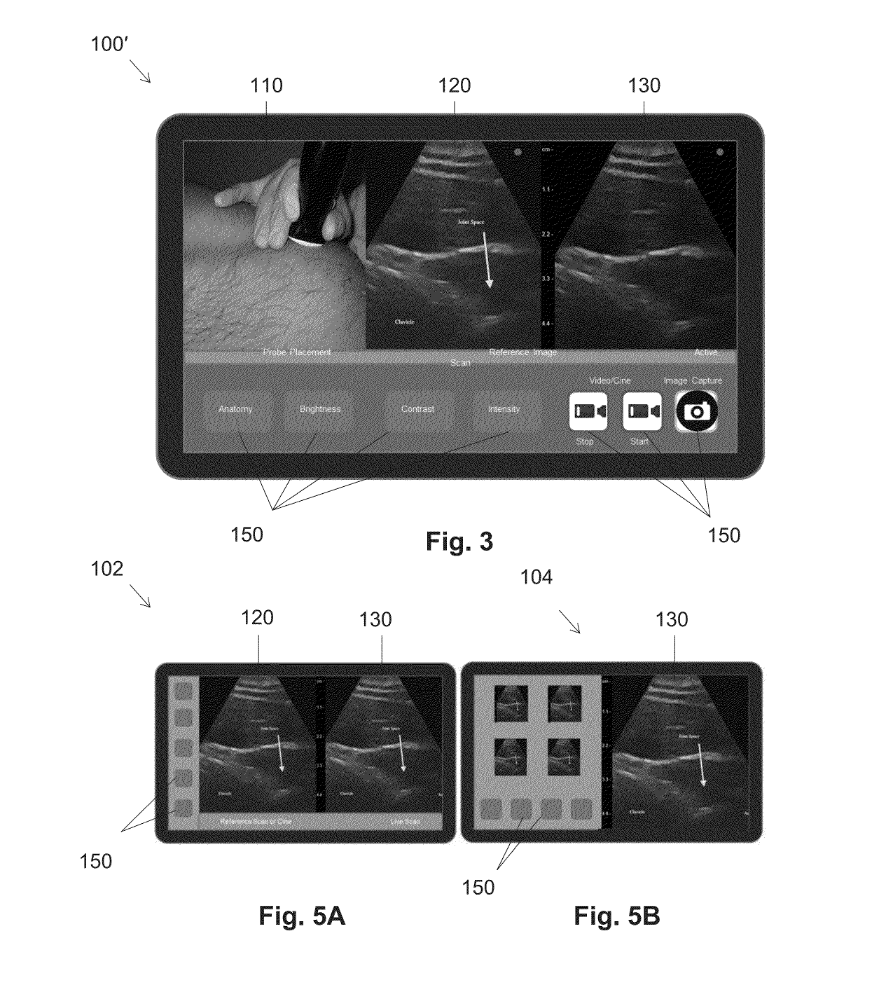

Specifically, FIG. 2 details a first UI arrangement 100, and FIG. 3 details another UI arrangement 100′. Common to each is a guidance image 110 showing idealized probe 20 placement relative to a patient in accordance with a selected procedure and/or approach. As shown in FIGS. 2 and 3, this image may be photographic/photorealistic. Or as shown in the far left image of UI 70 in FIG. 1, the guidance image may be presented as an illustration/cartoon or computer model.

FIGS. 2 and 3 also show a reference image 120 depicting what is expected of a real-time image 130 to be acquired by probe 20. Various labeling 140 may be applied to or overlaid upon the images 110/120/130 (e.g., as illustrated). Moreover, the reference view may show any or all time points intended for the procedure (e.g., as a still image that can be advanced frame-by-frame, as a looped clip or cine or otherwise). As such, the reference view may include an instrument pictured therein (such as needle 50) as its targeted position is intended in the probe-generated view 130. Any of these options provide the medical practitioner with various comparative options to successfully complete a medical procedure.

Additional notable features of the software may include:

automated billing in which a report is generated and submitted by system (patient info can be stored in system or network or removed once report is sent to billing); a language substitution table;

a graphical button substitution table (allowing graphics to be changed without programming);

positioning instructions (i.e., the probe placement image) can be video or still; the reference image can be cine or still (if cine, the cine is preferably the same length as positioning video, allowing synchronized instruction);

a procedure pick list populated from a table (where the table may include file names of positioning still or video and reference still or cine to be played/displayed);

programming so that functional modes (i.e., probe use and image capture) are only available if the table is populated;

programming so all settings are table driven by the selected procedure and not user adjustable;

programming to set gain, contrast or intensity by touching an icon(s) after which an adjustment slider(s) appears on the display screen then disappears after three seconds or other intuitive timeframe;

an option to illuminate joint/target in highlight color; and an ability to account for S, M, L, XL, XXL patient size, gender and/or form/obesity.

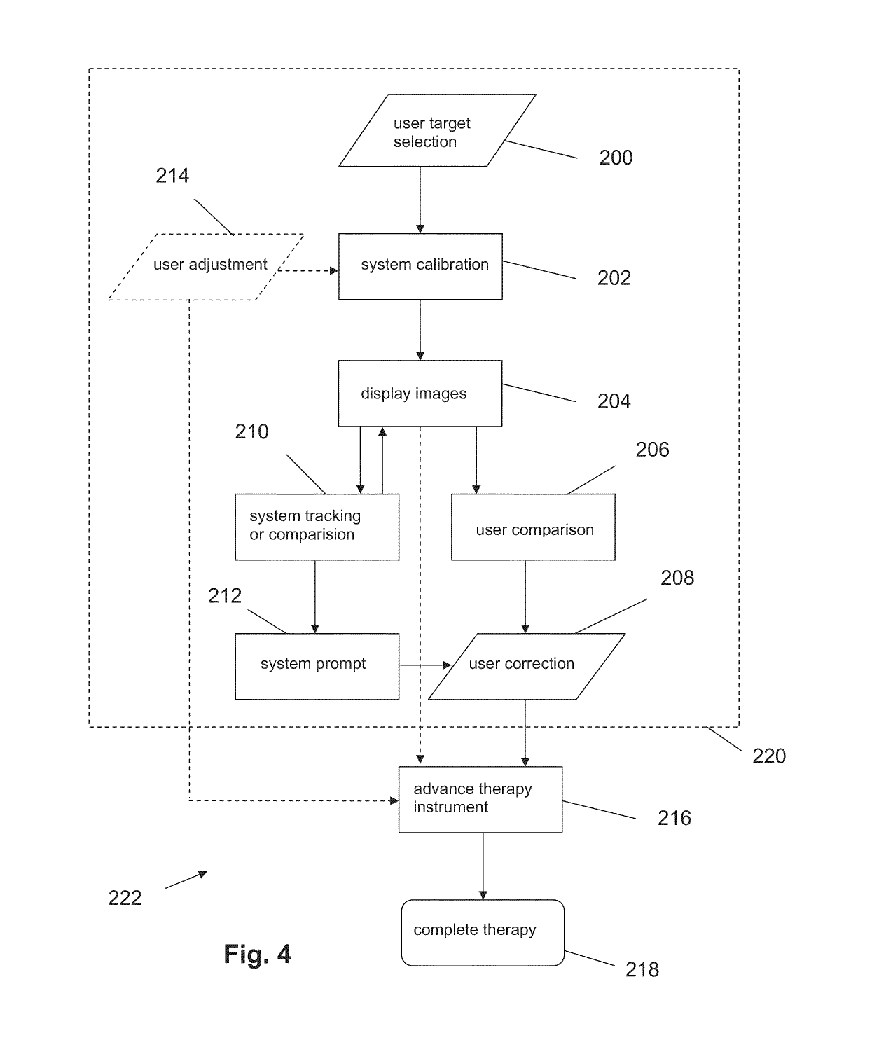

System use and operation is more generally illustrated in FIG. 4. At 200, the operator selects (e.g., from a menu or by keying-in a selection) a treatment target. For example, this may be a joint for steroid injection. At 202, the system may self-calibrate. At 204 the system displays the guidance and/or reference images. The probe image may also be displayed. At 206 the user may compare the actual probe position to the guidance image and/or the probe image to the reference image and take corrective action at 208. As another option, the system may (through tracking the 3D-position of the probe relative to a reference—such as table supporting the patient) check for proper probe positioning and at 210 either update the display images at 204 and/or prompt corrective positioning 212 for action by user at 208. As another option, such prompting or guidance and/or reference image update may be suggested by the system through feature-based comparison of a library of reference images against a current probe image. Software used in connection with such activity may include Scale Invariant Feature Keypoint Recognition Technology (SIFT) programming per U.S. Pat. No. 6,711,293. Before or after such activity, the user may adjust the system display settings, etc. at 214.

In completing a method of treatment 222, a physician (as the same or if different from the user) advances a therapy instrument under medical imaging into the subject/patient at 216. At 218, therapy is completed with the aspiration/removal of material or delivery of a therapeutic agent (such as a cortisone injection to a joint), wound dressing, etc. A subset of the methodology contemplated is the control and operation of the scanning system. This method 200 may be performed by a technician working as part of a team or by a physician performing the entire method 222.

It is further contemplated to include user sophistication selection/settings. A beginner mode may utilize all of the features above. An intermediate mode may turn off/disable the guidance image feature (as per the UI layout 102 shown in FIG. 5A). An expert mode may turn off both the guidance and the reference image (as per the UI layout 104 shown in FIG. 5B). Removing views as such may be desired to increase available display to increase active image size. Changes to the UI (such as repositioning selection, adjustment and/or action icons/buttons 150) may be desired as well.

Still, the subject software will incorporate the full optional functionality of the 3-screen approach (as exemplified by, but not limited to, that shown in FIGS. 1-3). Further optional features in a system or kit contemplated include alternate frequency probes as well as various supplies such as needles, gel and/or disposable offset pads.

### Variations

In addition to the embodiments that have been disclosed in detail above, still more are possible within the classes described and the inventors intend these to be encompassed within this Specification and claims. This disclosure is intended to be exemplary and the claims are intended to cover any modification or alternative that might be predictable to a person having ordinary skill in the art.

Accordingly, suitable image acquisition devices include: endoscope, arthroscope, X-Ray/fluoroscope, ultrasound transducer, MRI, and infrared to name (but not be limited to) several examples for “medical imaging” as referenced herein. In the same context, viewing systems employed may comprise CRT, LCD, DMD, DLP, Plasma, OLED, holographic, projection, etc. in the same context. Likewise, communications/information/data transmission between components may be any one of wire (such as ethernet, USB, serial, Thunderbolt, Lightning, etc.) or wireless (such as Bluetooth, InfraRed (IR), 802.11, cellular, Wi-Fi, etc.).

Moreover, the various illustrative processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a digital camera of any type including those using CMOS, CCD or other digital image capture technology.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

Operations as described herein can be carried out on or over a website. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The programs may be written inC, or Java, Brew or any other programming language. The programs may be resident on a storage medium, e.g., magnetic or optical, e.g., the computer hard drive, a removable disk or media such as a memory stick or SD media, or other removable medium. The programs may also be run over a network, for example, with a server or other machine sending signals to the local machine, which allows the local machine to carry out the operations described herein.

Also, it is contemplated that any optional feature of the embodiment variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language. All references cited are incorporated by reference in their entirety. Although the foregoing embodiments been described in detail for purposes of clarity of understanding, it is contemplated that certain modifications may be practiced within the scope of the appended claims.

What is claimed is:

1. A computer-implemented method of operating a scanning or imaging system that includes a scanning probe and a display, the scanning probe adapted for medical imaging of a subject, the method comprising:
selecting an imaging target site; and
showing on the display each of a guidance image for correct probe placement for visualizing the target site, a reference image corresponding to an expected view from the probe given correct probe placement, and a real-time image from the probe.

2. The method of claim 1, wherein probe position is tracked in three dimensions and the guidance image is updated to match the probe position.

3. The method of claim 1, wherein the guidance image displayed includes labeling.

4. The method of claim 1, wherein the labeling is selected from needle and anatomical landmarks.

5. The method of claim 1, wherein the guidance image for is a photograph.

6. The method of claim 1, wherein the guidance image is an illustration or model.

7. The method for claim 1, wherein at least one of the guidance image and reference image account for a physical characteristic of the subject.

8. The method of claim 7, wherein the characteristic is selected from gender and size.

9. The method of claim 1, wherein the guidance image is run as a movie clip or animation.

10. The method of claim 1, wherein the reference image is run as a cine.

11. A computer readable medium having stored thereon instructions, which when executed cause one or more processors to:
prompt user selection of a procedure target site;
based on the selection, be able to shown on the display each of a guidance image for correct probe placement for visualizing the target site, a reference image corresponds to an expected view from the probe given correct probe placement, and a real-time image from the probe; and
display at least the real-time image from the probe.

12. The computer readable medium of claim 11, wherein the instructions allow input of user selection to display only the real-time image.

13. The computer readable medium of claim 11, wherein the instructions allow input of user selection to display only the reference image and the real-time image.

14. The computer readable medium of claim 11, wherein the instructions allow input of user selection to display all of the guidance image, the reference image and the real-time image.

15. A method of medical treatment comprising:
selecting with a computer-based system a target site for treatment of a patient, viewing a guidance image on a display of the system;
positioning a medical imaging probe as indicated by the guidance image;
viewing a reference image on the display; and inserting an instrument into the subject; and
comparing a real-time image on the display generated by the probe with the reference Image.

16. The method of claim 15, wherein the selecting is by touching the display.

17. The method of claim 15, wherein the instrument is a needle.

18. The method of claim 17, further comprising injecting material into the patient.

19. The method of claim 18, further comprising comparing the injecting to a cine of injecting in the reference image.

20. The method of claim 15, wherein the real-time image is compared to a cine reference Image.
